(19) United States
(12) Patent Application Publication
(10) Pub. No.: US 2024/0148894 A1
(43) Pub. Date: May 9, 2024

(54) USE OF ANTIBODY-DRUG CONJUGATE TARGETING HER2 IN TREATMENT OF SPECIFIC BREAST CANCER

(71) Applicant: REMEGEN CO., LTD., Yantai (CN)

(72) Inventors: Jianmin FANG, Yantai (CN); Xiaohong SU, Yantai (CN); Xuguang GUO, Yantai (CN); Ruyi HE, Yantai (CN)

(21) Appl. No.: 18/450,966

(22) Filed: Aug. 16, 2023

Related U.S. Application Data
(63) Continuation of application No. PCT/CN2022/076554, filed on Feb. 17, 2022.

(30) Foreign Application Priority Data
Feb. 18, 2021 (CN) 202110189905.8
May 10, 2021 (CN) 202110506596.2

(51) Int. Cl. A61K 47/68; A61P 35/04

(52) U.S. Cl. CPC A61K 47/6855; A61K 47/68031; A61K 47/6889; A61P 35/04

(57) ABSTRACT

Provided is use of an antibody-drug conjugate targeting Her2 in the preparation of a medicine for treating breast cancer patients with liver metastasis or breast cancer patients without lung metastasis. Compared with a control drug group (lapatinib+capecitabine), the application of the antibody-drug conjugate provided in the treatment of breast cancer patients with liver metastasis and in the treatment of breast cancer patients without lung metastasis can significantly improve the survival time of the patients.

### CROSS REFERENCE TO RELATED APPLICATIONS

This application is a continuation application of PCT/CN2022/076554, filed Feb. 17, 2022, which claims the priority benefit of Chinese Application Nos. CN202110189905.8, filed Feb. 18, 2021, and CN202110506596.2, filed May 10 2021, each of which is incorporated herein by reference in its entirety.

### REFERENCE TO AN ELECTRONIC SEQUENCE LISTING

The contents of the electronic sequence listing (761682008201SEQLIST.xml; Size: 19,509 bytes; and Date of Creation: Jul. 31, 2023) is herein incorporated by reference in its entirety.

### FIELD

The present disclosure relates to the field of precise treatment of cancers, to use of an antibody-drug conjugate targeting HER2 (Human epidermal growth factor receptor 2) in the treatment of breast cancers with different biological behaviors, clinical indicators, and disease molecular types.

### BACKGROUND

As early as 1986, researchers raised the issue of Precision of Dosage (Coops W J. Precision of dosage [J]. Tijdschr Diergeneeskd, 1986, 111(2): 91). In 2010, American medical community proposed the concept of “Precision Medicine” (Shen B, Hwang J. The clinical utility of precision medicine: properly assessing the value of emerging diagnostic tests [j]. Clinical Pharmacology and Therapeutics, 2010, 88(6): 754-756). In 2015, the then U.S. president Obama proposed “Precision Medicine Initiative” in the State of the Union address, hoping to make cancer and other diseases achieve the goal of precise and individualized medications (Jackson D. Obama pushes ‘Precision Medicine Initiative’ [N]. USA TODAY, 2015-1-30). This is mainly because most current Medical Treatments are designed for “Average Patient”, and for the treatment of some patients, this “one-size-fits-all-approach” approach can be very successful, but not in other patients. Precision Medicine is also often understood as Personalized Medicine, in which “precision” in a broad sense refers to the most appropriate treatment for the right patient, i.e., it is necessary to determined clinically which therapeutic drugs are the most effective for a certain population, and which therapeutic drugs may be ineffective or bring greater toxic and side effects; “Personalized Information” is needed because each patient is unique, and then it is required clinically to make the correct classification of the specific diseases of specific patients and then give the correct and optimal therapeutic drugs, thereby providing more effective, safer and more economical medical services to the patients.

Breast cancer is a common malignant tumor in women. Due to changes in people's lifestyle concepts and ecological environment, the incidence of breast cancer is also increasing significantly. In the past 100 years, the treatment of breast cancer went through the proposal and promotion of breast conserving surgery, adjuvant therapy, endocrine therapy, targeted therapy, etc. The related clinical practice and clinical research results have accumulated a lot of experience and data for the treatment of the disease and improved the overall level of the diagnosis of the disease. However, the clinical treatment of breast cancer has always been based on histopathology, but when using the same regimen in breast cancer patients of the same pathological type and at the same clinical stage, the sensitivity of treatment and prognosis of patients vary greatly. Obviously, the traditional histopathological diagnosis and clinical staging can no longer well meet the development needs of tumor research. In fact, different breast cancer patients have different biological behaviors and clinical indicators, such as age, ethnicity, economic history, tumor family history, pathological type, pathological grade, tumor location, tumor size, lymph node metastasis, clinical stage and even disease molecular types. This is also the main reason for the obvious differences in treatment response, survival, and other aspects of breast cancers of the same histological type. Therefore, in clinical practice, the treatment should be classified according to patients' different biological behaviors, clinical indicators, and disease molecular types, etc., in order to achieve the purpose of precise personalized treatment (i.e., to give optimal therapeutic drugs according to individual differences).

Chinese patent publication no. CN105008398A discloses an antibody-drug conjugate (i.e., Disitamab vedotin) that can specifically bind to HER2 and has a drug moiety of MMAE. Currently, the drug is being explored as a treatment for various HER2-expressing (IHC 1+ or above) cancer indications including breast cancer, such as gastric and urothelial cancers, and HER2-low expressing (IHC 2+/FISH− or IHC 1+) cancer indications, such as HER2-low expressing breast cancer. In August 2020, the NMPA accepted the New Drug Application of Disitamab vedotin for the treatment of patients with locally advanced or metastatic gastric cancer (including gastroesophageal junction adenocarcinoma). In September of the same year, the U.S. FDA also granted a breakthrough therapy designation to Disitamab vedotin for the second-line treatment of HER2-expressing (IHC 2+ or IHC 3+) locally advanced or metastatic urothelial carcinoma indication. In addition, the new drug has also been granted Fast Track designation by the U.S. FDA for the treatment of urothelial carcinoma and gastric cancer.

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY

The present disclosure provides methods and uses for treating breast cancer patients with an anti-HER2 antibody-drug conjugate (ADC). These methods and uses are based at least in part on the in-depth analysis of clinical data presented herein, which demonstrate Applicant's surprising discovery that the ADC produced unexpected technical effects when being used for treating breast cancer patients with liver metastasis or breast cancer patients without lung metastasis. Compared with existing standard therapies, progression-free survival was significantly prolonged.

For example, in breast cancer patients with liver metastasis, the progression-free survival time in the Disitamab vedotin treatment group was 12.5 months, and the progression-free survival time in the capecitabine+lapatinib group was 5.6 months. In breast cancer patients without lung metastasis, the progression-free survival time in the Disitamab vedotin treatment group was 10.9 months and the progression-free survival time in the capecitabine+lapatinib group was 5.6 months. In contrast, the efficacy of the antibody-drug conjugates (ADC, especially Disitamab vedotin) provided by the present invention did not show a statistically significant advantage in overall sample of breast cancer patients and in a subgroup of breast cancer patients without bone metastasis or in a subgroup of breast cancer patients without viscera metastasis, and thus the superior results seen in breast cancer patients with liver metastasis or breast cancer patients without lung metastasis were surprising.

Overall, Disitamab vedotin effectively prolonged the disease progression-free time and survival time of breast cancer patients with liver metastasis or breast cancer patients without lung metastasis, thereby providing patients with more precise treatment options. That is to say, the application of the antibody-drug conjugates (ADC, especially Disitamab vedotin) provided by the present invention in the treatment of breast cancer patients with liver metastasis or the treatment of breast cancer patients without lung metastasis can achieve “precise treatment” for the corresponding patients. Compared with the control treatment group, the clinical application of Disitamab vedotin treatment group in breast cancer patients with liver metastasis and in breast cancer patients without lung metastasis has great significance and greatly prolongs the disease progression and possible survival time of the patients.

Provided herein is the use of an antibody-drug conjugate (ADC) in the preparation of a medicine for treating a breast cancer patient with liver metastasis, wherein the antibody-drug conjugate has the structure of the general formula Ab-(L-U)ₙ, wherein Ab represents anti-Her2 (Human epidermal growth factor receptor 2) antibody; L represents a linker; U represents a conjugated cytotoxic molecule; and n is an integer from 1 to 8, and represents the number of cytotoxic molecules bound to each antibody, and wherein: the antibody comprises a heavy chain variable region and a light chain variable region, where the CDR of the heavy chain variable region and/or the CDR of the light chain variable region have the same CDR sequences as Disitamab vedotin; the linker L comprises Maleimido-Caproyl-Valine-Citrulline-p-Aminobenzyloxy (mc-vc-pAB) and is covalently linked to the antibody by means of sulfhydryl conjugation, and the linking site is the interchain disulfide bond site of the antibody; and the cytotoxic molecule U comprises MMAE (monomethyl auristatin E). Further provided herein is the use of an antibody-drug conjugate (ADC) in the preparation of a medicine for treating of a breast cancer patient without lung metastasis, wherein the antibody-drug conjugate has the structure of the general formula Ab-(L-U)ₙ, wherein Ab represents anti-Her2 (Human epidermal growth factor receptor 2) antibody; L represents a linker; U represents a conjugated cytotoxic molecule; and n is an integer from 1 to 8, and represents the number of cytotoxic molecules bound to each antibody, and wherein: the antibody comprises a heavy chain variable region and a light chain variable region, where the CDR of the heavy chain variable region and/or the CDR of the light chain variable region have the same CDR sequences as Disitamab vedotin; the linker L comprises Maleimido-Caproyl-Valine-Citrulline-p-Aminobenzyloxy (mc-vc-pAB) and is covalently linked to the antibody by means of sulfhydryl conjugation, and the linking site is the interchain disulfide bond site of the antibody; and the cytotoxic molecule U comprises MMAE (monomethyl auristatin E).

In some embodiments, the breast cancer patient is positive for HER2 expression. In some embodiments, a sample obtained from the breast cancer of the patient is HER2 positive. In some embodiments, the sample obtained from the breast cancer of the patient is HER2 positive based on a fluorescence in situ hybridization (FISH) assay (FISH) and/or immunohistochemistry (IHC) assay. In some embodiments, HER2 expression in the sample obtained from the breast cancer of the patient is: IHC3+; IHC2+ or IHC3+; IHC2+ or FISH+; IHC3+ or FISH+; IHC2+ and FISH+; IHC3+ and FISH+; or IHC3+ and FISH− or not detected. In some embodiments, a sample obtained from the breast cancer of the patient is estrogen receptor (ER) positive and/or progesterone receptor (PR) positive. In some embodiments, a sample obtained from the breast cancer of the patient is estrogen receptor (ER) positive or progesterone receptor (PR) positive. In some embodiments, a sample obtained from the breast cancer of the patient is ER negative and PR negative. In some embodiments, the patient has locally advanced or metastatic breast cancer. In some embodiments, the patient has stage IV breast cancer. In some embodiments, the patient has unresectable breast cancer.

In some embodiments, the antibody comprises a heavy chain variable (VH) region and a light chain variable (VL) region; wherein the VH region comprises an HCDR1 comprising the amino acid sequence of GYTFTDYY (SEQ ID NO:3), an HCDR2 comprising the amino acid sequence of VNPDHGDS (SEQ ID NO:4), and an HCDR3 comprising the amino acid sequence of ARNYLFDH (SEQ ID NO:5); and wherein the VL region comprises a LCDR1 comprising the amino acid sequence of QDVGTA (SEQ ID NO:6), a LCDR2 comprising the amino acid sequence of WAS (SEQ ID NO:7), and a LCDR3 comprising the amino acid sequence of HQFATYT (SEQ ID NO:8). In some embodiments, the antibody comprises a heavy chain variable (VH) region and a light chain variable (VL) region; wherein the VH region comprises an HCDR1 comprising the amino acid sequence of DYYIH (SEQ ID NO:11), an HCDR2 comprising the amino acid sequence of RVNPDHGDSYYNQKFKD (SEQ ID NO:12), and an HCDR3 comprising the amino acid sequence of ARNYLFDHW (SEQ ID NO:13); and wherein the VL region comprises a LCDR1 comprising the amino acid sequence of KASQDVGTAVA (SEQ ID NO:14), a LCDR2 comprising the amino acid sequence of WASIRHT (SEQ ID NO:15), and a LCDR3 comprising the amino acid sequence of HQFATYT (SEQ ID NO:8). In some embodiments, the antibody comprises a heavy chain variable (VH) region and a light chain variable (VL) region; wherein the VH region comprises the amino acid sequence of EVQLVQSGAEVKKPGATVKISCKVSGYTFTDYYIHWVQQAPGKGLEWMGRVNPDH GDSYYNQKFKDKATITADKSTDTAYMELSSLRSEDTAVYFCARNYLFDHWGQGTL VTVSS (SEQ ID NO:9); and/or wherein the VL region comprises the amino acid sequence of DIQMTQSPSSVSASVGDRVTITCKASQDVGTAVAWYQQKPGKAPKLLIYWASIRHT GVPSRFSGSGSGTDFTLTISSLQPEDFATYYCHQFATYTFGGGTKVEIK (SEQ ID NO:10). In some embodiments, the antibody comprises a heavy chain variable (VH) region and a light chain variable (VL) region; wherein the VH region comprises the amino acid sequence of EVQLVQSGAEVKKPGATVKISCKVSGYTFTDYYIHWVQQAPGKGLEWMGRVNPDH GDSYYNQKFKDKATITADKSTDTAYMELSSLRSEDTAVYFCARNYLFDHWGQGTL VTVSS (SEQ ID NO:9); and wherein the VL region comprises the amino acid sequence of DIQMTQSPSSVSASVGDRVTITCKASQDVGTAVAWYQQKPGKAPKLLIYWASIRHT GVPSRFSGSGSGTDFTLTISSLQPEDFATYYCHQFATYTFGGGTKVEIK (SEQ ID NO:10). In some embodiments, the antibody is a murine, chimeric, or humanized antibody. In some embodiments, the antibody is a human IgG antibody. In some embodiments, the antibody is a human IgG1, IgG2, or IgG4 antibody. In some embodiments, the amino acid sequence of the heavy chain of the antibody is shown in SEQ ID NO:1, and the amino acid sequence of the light chain of the antibody is shown in SEQ ID NO:2. In some embodiments, the amino acid sequence of the heavy chain of the antibody is shown in SEQ ID NO:1 without the C-terminal lysine, and the amino acid sequence of the light chain of the antibody is shown in SEQ ID NO:2.

In some embodiments, the antibody-drug conjugate is Disitamab vedotin or a biosimilar thereof. In some embodiments, the average DAR (i.e., Drug-to-Antibody Ratio) value of the antibody-drug conjugate is any number from 2 to 7. In some embodiments, the average DAR value is 4±0.5.

In some embodiments, the patient has previously received one or more prior treatments of chemotherapy drugs, targeted therapy, immunotherapy, and endocrine therapy. In some embodiments, the patient has previously received taxane systemic therapy. In some embodiments, the patient has previously received systemic therapy with trastuzumab or a biosimilar thereof at least once. In some embodiments, the medicine is to be administered intranasally, subcutaneously, intradermally, intramuscularly or intravenously. In some embodiments, the medicine is to be administered at a dose of 2.0 mg/kg every 2 weeks. In some embodiments, the medicine is to be administered as a monotherapy. In some embodiments, administration of the antibody-drug conjugate to the breast cancer patient results in improved progression-free survival (PFS), as compared to administration of capecitabine and lapatinib.

Further provided herein are methods of treating breast cancer, comprising administering to a patient in need thereof a therapeutically effective amount of an antibody-drug conjugate (ADC), wherein the antibody-drug conjugate has the structure of the general formula Ab-(L-U)ₙ, wherein Ab represents an antibody that specifically binds human epidermal growth factor receptor 2 (HER2); L represents a linker; U represents a cytotoxic molecule; and n is an integer from 1 to 8 representing a number of cytotoxic molecule(s) conjugated to each antibody, and wherein: the antibody comprises a heavy chain variable region and a light chain variable region, where the CDR of the heavy chain variable region and/or the CDR of the light chain variable region have the same CDR sequences as Disitamab vedotin; the linker L comprises Maleimido-Caproyl-Valine-Citrulline-p-Aminobenzyloxy (mc-vc-pAB) and is covalently linked to the antibody by means of sulfhydryl conjugation, and the linking site is the interchain disulfide bond site of the antibody; the cytotoxic molecules U comprise MMAE (monomethyl auristatin E). In some embodiments, the patient has a liver metastasis. In some embodiments, the patient does not have a lung metastasis. In some embodiments, the patient has a liver metastasis and does not have a lung metastasis.

In some embodiments, a sample obtained from the breast cancer of the patient is HER2 positive. In some embodiments, the breast cancer expresses HER2, e.g., overexpresses HER2. In some embodiments, a sample obtained from the breast cancer of the patient is HER2 positive based on a fluorescence in situ hybridization (FISH) assay (FISH) and/or immunohistochemistry (IHC) assay. In some embodiments, HER2 expression in the sample obtained from the breast cancer of the patient is: IHC3+; IHC2+ or IHC3+; IHC2+ or FISH+; IHC3+ or FISH+; IHC2+ and FISH+; IHC3+ and FISH+; or IHC3+ and FISH− or not detected. In some embodiments, a sample obtained from the breast cancer of the patient is estrogen receptor (ER) positive and/or progesterone receptor (PR) positive. In some embodiments, a sample obtained from the breast cancer of the patient is estrogen receptor (ER) positive and progesterone receptor (PR) positive. In some embodiments, a sample obtained from the breast cancer of the patient is ER negative and PR negative.

In some embodiments, the patient has locally advanced or metastatic breast cancer. In some embodiments, the patient has stage IV breast cancer. In some embodiments, the patient has unresectable breast cancer. In some embodiments, the breast cancer is infiltrating locally advanced or metastatic breast cancer as established by histology and/or cytology and is unresectable.

In some embodiments, the antibody comprises a heavy chain variable (VH) region and a light chain variable (VL) region; wherein the VH region comprises an HCDR1 comprising the amino acid sequence of GYTFTDYY (SEQ ID NO:3), an HCDR2 comprising the amino acid sequence of VNPDHGDS (SEQ ID NO:4), and an HCDR3 comprising the amino acid sequence of ARNYLFDH (SEQ ID NO:5); and/or wherein the VL region comprises a LCDR1 comprising the amino acid sequence of QDVGTA (SEQ ID NO:6), a LCDR2 comprising the amino acid sequence of WAS (SEQ ID NO:7), and a LCDR3 comprising the amino acid sequence of HQFATYT (SEQ ID NO:8). In some embodiments, the antibody comprises a heavy chain variable (VH) region and a light chain variable (VL) region; wherein the VH region comprises an HCDR1 comprising the amino acid sequence of GYTFTDYY (SEQ ID NO:3), an HCDR2 comprising the amino acid sequence of VNPDHGDS (SEQ ID NO:4), and an HCDR3 comprising the amino acid sequence of ARNYLFDH (SEQ ID NO:5); and wherein the VL region comprises a LCDR1 comprising the amino acid sequence of QDVGTA (SEQ ID NO:6), a LCDR2 comprising the amino acid sequence of WAS (SEQ ID NO:7), and a LCDR3 comprising the amino acid sequence of HQFATYT (SEQ ID NO:8). In some embodiments, the antibody comprises a heavy chain variable (VH) region and a light chain variable (VL) region; wherein the VH region comprises an HCDR1 comprising the amino acid sequence of DYYIH (SEQ ID NO:11), an HCDR2 comprising the amino acid sequence of RVNPDHGDSYYNQKFKD (SEQ ID NO:12), and an HCDR3 comprising the amino acid sequence of ARNYLFDHW (SEQ ID NO:13); and/or wherein the VL region comprises a LCDR1 comprising the amino acid sequence of KASQDVGTAVA (SEQ ID NO:14), a LCDR2 comprising the amino acid sequence of WASIRHT (SEQ ID NO:15), and a LCDR3 comprising the amino acid sequence of HQFATYT (SEQ ID NO:8). In some embodiments, the antibody comprises a heavy chain variable (VH) region and a light chain variable (VL) region; wherein the VH region comprises an HCDR1 comprising the amino acid sequence of DYYIH (SEQ ID NO:11), an HCDR2 comprising the amino acid sequence of RVNPDHGDSYYNQKFKD (SEQ ID NO:12), and an HCDR3 comprising the amino acid sequence of ARNYLFDHW (SEQ ID NO:13); and wherein the VL region comprises a LCDR1 comprising the amino acid sequence of KASQDVGTAVA (SEQ ID NO:14), a LCDR2 comprising the amino acid sequence of WASIRHT (SEQ ID NO:15), and a LCDR3 comprising the amino acid sequence of HQFATYT (SEQ ID NO:8). In some embodiments, the antibody comprises a heavy chain variable (VH) region and a light chain variable (VL) region; wherein the VH region comprises the amino acid sequence of EVQLVQSGAEVKKPGATVKISCKVSGYTFTDYYIHWVQQAPGKGLEWMGRVNPDH GDSYYNQKFKDKATITADKSTDTAYMELSSLRSEDTAVYFCARNYLFDHWGQGTL VTVSS (SEQ ID NO:9); and/or wherein the VL region comprises the amino acid sequence of DIQMTQSPSSVSASVGDRVTITCKASQDVGTAVAWYQQKPGKAPKLLIYWASIRHT GVPSRFSGSGSGTDFTLTISSLQPEDFATYYCHQFATYTFGGGTKVEIK (SEQ ID NO:10). In some embodiments, the antibody comprises a heavy chain variable (VH) region and a light chain variable (VL) region; wherein the VH region comprises the amino acid sequence of EVQLVQSGAEVKKPGATVKISCKVSGYTFTDYYIHWVQQAPGKGLEWMGRVNPDH GDSYYNQKFKDKATITADKSTDTAYMELSSLRSEDTAVYFCARNYLFDHWGQGTL VTVSS (SEQ ID NO:9); and wherein the VL region comprises the amino acid sequence of DIQMTQSPSSVSASVGDRVTITCKASQDVGTAVAWYQQKPGKAPKLLIYWASIRHT GVPSRFSGSGSGTDFTLTISSLQPEDFATYYCHQFATYTFGGGTKVEIK (SEQ ID NO:10). In some embodiments, the antibody is a human IgG antibody, e.g., a human IgG1, IgG2, or IgG4 antibody. In some embodiments, the antibody comprises a human Fc region, e.g., a human IgG1, IgG2, or IgG4 Fc region. In some embodiments, the antibody comprises a heavy chain comprising the amino acid sequence of SEQ ID NO:1 and a light chain comprising the amino acid sequence of SEQ ID NO:2. In some embodiments, the antibody comprises a heavy chain comprising the amino acid sequence of SEQ ID NO:1 without the C-terminal lysine and a light chain comprising the amino acid sequence of SEQ ID NO:2.

In some embodiments, the antibody-drug conjugate is Disitamab vedotin or a biosimilar thereof. In some embodiments, the average Drug-to-Antibody Ratio (DAR) of the antibody-drug conjugate is any number from 2 to 7, e.g., the average DAR value is 4±0.5.

In some embodiments, prior to administration of the ADC, the patient has previously received one or more prior treatments comprising a chemotherapeutic agent, targeted therapy, immunotherapy, or endocrine therapy. In some embodiments, prior to administration of the ADC, the patient has received a taxane systemic therapy. In some embodiments, prior to administration of the ADC, the patient has received systemic therapy with trastuzumab or a biosimilar thereof at least once. In some embodiments, prior to administration of the ADC, the patient has received systemic therapy with an anti-HER2 antibody at least once. In some embodiments, the ADC is administered intranasally, subcutaneously, intradermally, intramuscularly, or intravenously. In some embodiments, the ADC is administered at a dose of 2.0 mg/kg every 2 weeks. In some embodiments, the ADC is administered as a monotherapy. In some embodiments, administration of the ADC results in improved progression-free survival (PFS) of the patient, as compared to administration of capecitabine and lapatinib. In some embodiments, the ADC is administered in a pharmaceutical composition comprising the ADC and a pharmaceutically acceptable carrier.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 is a schematic diagram of the structure of monomethyl auristatin E (MMAE).

FIG. 2 is a schematic diagram of exemplary structures of the antibody-drug conjugates of the structural general formula Ab-(L-U)ₙ of the present disclosure under one of the possible conjugation conditions (L is linked to one or more interchain disulfide bond sites of the antibody through sulfhydryl conjugation) where n is 1, 2, 3, 4, 5, 6, 7, and 8, respectively; L is Maleimido-Caproyl-Valine-Citrulline-p-Aminobenzyloxy (mc-vc-pAB); U is MMAE; and the structure of “-L-U” is as follows

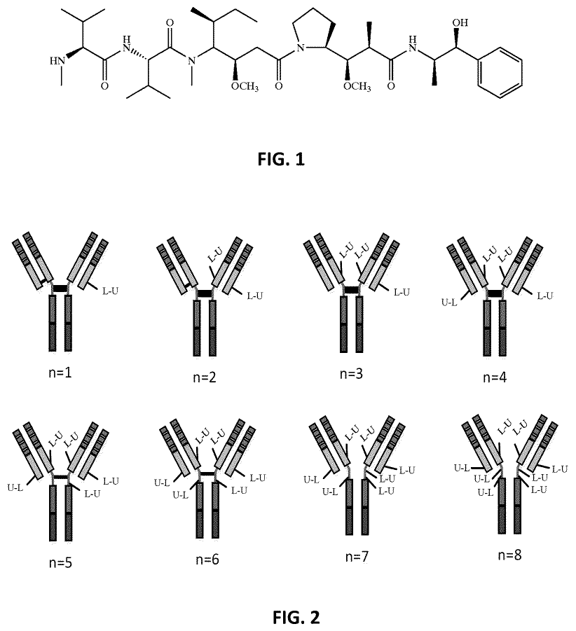

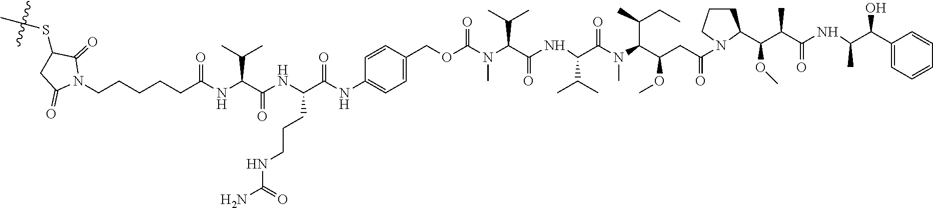 [SMILES: *SC1CC(=O)N(CCCCCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)C1=O]

### DETAILED DESCRIPTION

### I. Definitions

Unless otherwise defined, all technical and scientific terms used herein have the same meaning as understood by those of ordinary skill in the art. For definitions and terms in the field, professionals can refer to Current Protocols in Molecular Biology (Ausubel).

The three-letter and one-letter codes for amino acids used in the present invention are as described in J. biol. chem, 243, p 3558 (1968).

In the present invention, the determination or numbering method of the complementary determining regions (CDRs) of the variable domains of antibodies includes IMGT, Kabat, Chothia, AbM, and Contact methods which are well known in the art.

The “antibody” used in the present invention encompasses a variety of antibody structures including, but not limited to, monoclonal antibodies, polyclonal antibodies, multispecific antibodies (e.g., bispecific antibodies), and antigen binding fragments. “Antigen binding fragment” used in the present invention refers to an antibody fragment comprising a heavy chain variable region or a light chain variable region of the antibody and being sufficient to retain the same binding specificity as its source antibody and sufficient affinity. In particular, the antigen binding fragments comprise Fab, F(ab′), and F(ab′)2, which contain at least one immunoglobulin fragment sufficient to make a specific antigen bind to the polypeptide. The above fragments can be prepared by synthesis, or by an enzymic method, or by chemical cutting of intact immunoglobulin, or genetically engineered by using recombinant DNA techniques. The production methods of the above fragments are well known in the art.

The term “murine antibody” as used in the present invention is a monoclonal antibody prepared according to the knowledge and skill in the art. During preparation, a corresponding antigen was injected into the test subjects, and then hybridomas expressing an antibody having the desired sequence or functional characteristics were isolated. In a preferred embodiment of the invention, the murine antibody or antigen binding fragment thereof can further comprise a light chain constant region of murine κ or λ chain or a variant thereof, or further comprises a heavy chain constant region of murine IgG1, IgG2, IgG3, or a variant thereof.

The term “chimeric antibody” as used in the present invention is an antibody that is a fusion of a variable region of a murine antibody with a constant region of a human antibody and can reduce immune response induced by the murine antibody. When establishing the chimeric antibody, hybridomas which secrete a murine specific monoclonal antibody are first established. Then, variable region genes are cloned from murine hybridoma cells, and as required, constant region genes are cloned from the human antibody. The mouse variable region genes and the human constant region genes are linked to form a chimeric gene and inserted into a human vector. Finally, chimeric antibody molecules are expressed in a eukaryotic industrial system or a prokaryotic industrial system. In a preferred embodiment of the invention, the antibody light chain of the chimeric antibody further comprises a light chain constant region of human κ or λ chain or a variant thereof. The antibody heavy chain of the chimeric antibody further comprises a heavy chain constant region of human IgG1, IgG2, IgG3, IgG4, or a variant thereof. The constant region of the human antibody can be selected from the heavy chain constant region of human IgG1, IgG2, IgG3, or IgG4, or a variant thereof, and preferably comprise the heavy chain constant region of human IgG2 or IgG4. Alternatively, IgG4 which has no ADCC toxicity (antibody-dependent cell-mediated cytotoxicity) after an amino acid mutation occurred is used.

The term “humanized antibody” as used in the present invention, also known as CDR-grafted antibody, refers to an antibody generated by grafting of a mouse CDR sequence into human antibody variable region framework (i.e., human germline antibody framework sequences of different types). It comprises a CDR region derived from a non-human antibody and the rest of the antibody molecule is derived from one human antibody (or several human antibodies). Furthermore, in order to preserve binding affinity, some residues of the framework region (referred to as FR) segments can be modified (Jones et al., Nature, 321:522-525, 1986; Verhoeyen et al., Science, 239:1534-1536, 1988; and Riechmann et al., Nature, 332:323-327, 1988). The humanized antibodies or fragments thereof according to the invention can be prepared by techniques known to those skilled in the art (e.g., as described in Singer et al., J. Immun. 150:2844-2857, 1992; Mountain et al., Biotechnol. Genet. Eng. Rev., 10: 1-142, 1992; or Bebbington et al., Bio/Technology, 10: 169-175, 1992).

The term average “DAR” value as used in the present invention, namely the Drug-to-Antibody Ratio, refers to the average value of the number of drugs linked to the antibody in the antibody-drug conjugate preparation.

The term “sulfhydryl conjugation” as used in the present invention refers to a conjugation means by which the linker is covalently linked to a free sulfhydryl group on the antibody. Cysteine exists in the form of a disulfide bond in the antibody, and there are 4 pairs of interchain disulfide bonds in an IgG antibody, which are easy to be reduced. Therefore, during the preparation of the antibody-drug conjugate, the 4 pairs of interchain disulfide bonds in the IgG antibody are often reduced, which produces the above-mentioned “free sulfhydryl group on the antibody”. Moreover, it is just because there are 4 pairs of interchain disulfide bonds in the IgG antibody, and when they are reduced, a maximum of 8 free sulfhydryl groups will be generated, an IgG antibody will have a maximum of 8 sulfhydryl conjugation sites. Thus, When n in the antibody-drug conjugate of the general formula Ab-(L-U)ₙ is 1, “L-U” can be covalently linked to any 1 site of the 8 sulfhydryl conjugation sites; similarly, when n is 2, “L-U” can be covalently linked to any 2 sites of the 8 sulfhydryl conjugation sites; when n is 3, “L-U” can be linked to any 3 sites of the 8 sulfhydryl conjugation sites; when n is 4, “L-U” can be covalently linked to any 4 sites of the 8 sulfhydryl conjugation sites; when n is 5, “L-U” can be covalently linked to any 5 sites of the 8 sulfhydryl conjugation sites; when n is 6, “L-U” can be covalently linked to any 6 sites of the 8 sulfhydryl conjugation sites; when n is 7, “L-U” can be covalently linked to any 7 sites of the 8 sulfhydryl conjugation sites; and when n is 8, “L-U” can be covalently linked to the 8 sulfhydryl conjugation sites.

### II. Uses and Methods

Certain aspects of the present disclosure relate to antibody-drug conjugates that bind HER2 (as well as methods and uses for the same). In some embodiments, the antibody-drug conjugate involved has the structure of the general formula Ab-(L-U)n, where Ab represents anti-HER2 (Human epidermal growth factor receptor 2) antibody; L represents a linker; U represents conjugated cytotoxic molecules; and n is an integer from 1 to 8 (such as 1, 2, 3, 4, 5, 6, 7, or 8), and represents the number of cytotoxic molecules bound to each antibody.

In some embodiments, the cytotoxic molecule is an auristatin, or an analog or derivative thereof. Auristatins are derivatives of the natural product dolastatin. Exemplary auristatins include dolostatin-10, auristatin E, auristatin T, MMAE (N-methylvaline-valine-dolaisoleuine-dolaproine-norephedrine or monomethyl auristatin E) and MMAF (N-methylvaline-valine-dolaisoleuine-dolaproine-phenylalanine or dovaline-valine-dolaisoleunine-dolaproine-phenylalanine), AEB (ester produced by reacting auristatin E with paraacetyl benzoic acid), AEVB (ester produced by reacting auristatin E with benzoylvaleric acid), and AFP (dimethylvaline-valine-dolaisoleuine-dolaproine-phenylalanine-p-phenylenediamine or auristatin phenylalanine phenylenediamine) WO 2015/057699 describes PEGylated auristatins including MMAE. Additional dolostatin derivatives contemplated for use are disclosed in U.S. Pat. No. 9,345,785, incorporated herein by reference for any purpose.

In some embodiments, the cytotoxic molecule is MMAE. In other embodiments, the cytotoxic agent is MMAF.

In some embodiments, the anti-HER2 (Human epidermal growth factor receptor 2) antibody or the functional fragment thereof in the antibody-drug conjugate provided by the present invention comprises a heavy chain variable region and a light chain variable region, where the CDR of the heavy chain variable region and/or the CDR of the light chain variable region have the same CDR sequences as Disitamab vedotin; the linker L comprises Maleimido-Caproyl-Valine-Citrulline-p-Aminobenzyloxy (mc-vc-pAB); and the cytotoxic molecules U comprise MMAE (monomethyl auristatin E).

In some embodiments, the linker L is covalently linked to the antibody by means of sulfhydryl conjugation, and the linking site is the interchain disulfide bond site of the antibody.

In some embodiments, the antibody-drug conjugates of the present invention is a mixture of antibody-drug conjugates linked with 2-7 cytotoxic molecules, where the average DAR (i.e., Drug-to-Antibody Ratio) value of the antibody-drug conjugates is any number from 2 to 7; more preferably, the average DAR value of the antibody-drug conjugates of the present invention is approximately equal to 2, 3, 4, 5, 6, or 7. In some specific examples of the present invention, the average DAR value of the antibody-drug conjugates of the present invention is 4±0.5.

In some embodiments, the corresponding CDRs 1-3 of the heavy chain variable regions and the light chain variable region of the anti-HER2 antibody involved in the present invention are as follows (IMGT numbering):

| | | |
|---|---|---|
| HCDR1: | GYTFTDYY | SEQ ID NO: 3 |
| HCDR2: | VNPDHGDS | SEQ ID NO: 4 |
| HCDR3: | ARNYLFDH | SEQ ID NO: 5 |
| LCDR1: | QDVGTA | SEQ ID NO: 6 |
| LCDR2: | WAS | SEQ ID NO: 7 |
| LCDR3: | HQFATYT | SEQ ID NO: 8 |

In some embodiments, the corresponding CDRs 1-3 of the heavy chain variable regions and the light chain variable region of the anti-HER2 antibody involved in the present invention are as follows:

| | | |
|---|---|---|
| HCDR1: | DYYIH | SEQ ID NO: 11 |
| HCDR2: | RVNPDHGDSYYNQKFKD | SEQ ID NO: 12 |
| HCDR3: | ARNYLFDHW | SEQ ID NO: 13 |
| LCDR1: | KASQDVGTAVA | SEQ ID NO: 14 |
| LCDR2: | WASIRHT | SEQ ID NO: 15 |
| LCDR3: | HQFATYT | SEQ ID NO: 8 |

In some embodiments, the anti-HER2 antibody comprises the corresponding CDRs 1-3 of the heavy chain variable regions and the light chain variable region represented by SEQ ID Nos:3-8, but with 1, 2, or 3 substitutions (e.g., conservative substitutions), insertions, or deletions relative to SEQ ID Nos:3-8, but an anti-HER2 antibody comprising that sequence retains the ability to bind to HER2. In some embodiments, the anti-HER2 antibody comprises the corresponding CDRs 1-3 of the heavy chain variable regions and the light chain variable region represented by SEQ ID Nos:11-15 and 8, but with 1, 2, or 3 substitutions (e.g., conservative substitutions), insertions, or deletions relative to SEQ ID Nos: 11-15 and 8, but an anti-HER2 antibody comprising that sequence retains the ability to bind to HER2.

In some embodiments, the anti-HER2 (Human epidermal growth factor receptor 2) antibody in the antibody-drug conjugate provided by the present invention is murine, chimeric, humanized or fully human, preferably a humanized monoclonal antibody. In some embodiments, the antibody is a monoclonal antibody.

In some embodiments, the anti-HER2 (Human epidermal growth factor receptor 2) antibody in the antibody-drug conjugate provided by the present invention is IgG, including IgG1, IgG2, IgG3, and IgG4, and more preferably IgG1, IgG2, and IgG4.

In some embodiments, the antibody comprises a heavy chain variable (VH) region and a light chain variable (VL) region; wherein the VH region comprises an amino acid sequence with at least 90%, at least 91%, at least 92%, at least 93%, at least 94%, at least 95%, at least 96%, at least 97%, at least 98%, at least 99%, or 100% identity to the sequence EVQLVQSGAEVKKPGATVKISCKVSGYTFTDYYIHWVQQAPGKGLEWMGRVNPDH GDSYYNQKFKDKATITADKSTDTAYMELSSLRSEDTAVYFCARNYLFDHWGQGTL VTVSS (SEQ ID NO:9); and/or wherein the VL region comprises an amino acid sequence with at least 90%, at least 91%, at least 92%, at least 93%, at least 94%, at least 95%, at least 96%, at least 97%, at least 98%, at least 99%, or 100% identity to the sequence DIQMTQSPSSVSASVGDRVTITCKASQDVGTAVAWYQQKPGKAPKLLIYWASIRHT GVPSRFSGSGSGTDFTLTISSLQPEDFATYYCHQFATYTFGGGTKVEIK (SEQ ID NO:10). In certain embodiments, the VH sequence (e.g., having at least 90%, 91%, 92%, 93%, 94%, 95%, 96%, 97%, 98%, or 99% identity to SEQ ID NO:9) contains substitutions (e.g., conservative substitutions), insertions, or deletions relative to SEQ ID NO:9, but an anti-HER2 antibody comprising that sequence retains the ability to bind to HER2. In certain embodiments, a total of 1 to 10 amino acids have been substituted, inserted and/or deleted in SEQ ID NO: 9. In certain embodiments, substitutions, insertions, or deletions occur in regions outside the CDRs (i.e., in the FRs). In certain embodiments, the VL sequence (e.g., having at least 90%, 91%, 92%, 93%, 94%, 95%, 96%, 97%, 98%, or 99% identity to SEQ ID NO:10) contains substitutions (e.g., conservative substitutions), insertions, or deletions relative to SEQ ID NO:10, but an anti-HER2 antibody comprising that sequence retains the ability to bind to HER2. In certain embodiments, a total of 1 to 10 amino acids have been substituted, inserted and/or deleted in SEQ ID NO: 10. In certain embodiments, substitutions, insertions, or deletions occur in regions outside the CDRs (i.e., in the FRs).

In some embodiments, the antibody comprises a heavy chain variable (VH) region and a light chain variable (VL) region; wherein the VH region comprises the amino acid sequence of EVQLVQSGAEVKKPGATVKISCKVSGYTFTDYYIHWVQQAPGKGLEWMGRVNPDH GDSYYNQKFKDKATITADKSTDTAYMELSSLRSEDTAVYFCARNYLFDHWGQGTL VTVSS (SEQ ID NO:9); and wherein the VL region comprises the amino acid sequence of DIQMTQSPSSVSASVGDRVTITCKASQDVGTAVAWYQQKPGKAPKLLIYWASIRHT GVPSRFSGSGSGTDFTLTISSLQPEDFATYYCHQFATYTFGGGTKVEIK (SEQ ID NO:10).

In some embodiments, the antibody-drug conjugate of the present invention is Disitamab vedotin, which is an antibody-drug conjugate targeting a HER2 target, where the linker moiety L is Maleimido-Caproyl-Valine-Citrulline-p-Aminobenzyloxy (mc-vc-pAB); the cytotoxic molecules U comprise MMAE (monomethyl auristatin E); the linker L is covalently linked to the antibody by means of sulfhydryl conjugation; and the average DAR value is 4±0.5.

In some embodiments, the heavy chain amino acid sequence of the antibody Ab in the antibody-drug conjugate involved in the present invention is shown in SEQ ID NO: 1, and the light chain amino acid sequence thereof is shown in SEQ ID NO: 2. In some embodiments, the heavy chain comprises the amino acid sequence of SEQ ID NO:1 without the C-terminal lysine.

| Heavy chain amino acid sequence- | |
|---|---|
| SEQ ID NO: 1 | |
| EVQLVQSGAE VKKPGATVKI SCKVSGYTFT DYYIHWVQQA PGKGLEWMGR | 50 |
| VNPDHGDSYY NQKFKDKATI TADKSTDTAY MELSSLRSED TAVYFCARNY | 100 |
| LFDHWGQGTL VTVSSASTKG PSVFPLAPSS KSTSGGTAAL GCLVKDYFPE | 150 |
| PVTVSWNSGA LTSGVHTFPA VLQSSGLYSL SSVVTVPSSS LGTQTYICNV | 200 |
| NHKPSNTKVD KKVEPKSCDK THTCPPCPAP ELLGGPSVFL FPPKPKDTLM | 250 |
| ISRTPEVTCV VVDVSHEDPE VKFNWYVDGV EVHNAKTKPR EEQYNSTYRV | 300 |
| VSVLTVLHQD WLNGKEYKCK VSNKALPAPI EKTISKAKGQ PREPQVYTLP | 350 |
| PSREEMTKNQ VSLTCLVKGF YPSDIAVEWE SNGQPENNYK TTPPVLDSDG | 400 |
| SFFLYSKLTV DKSRWQQGNV FSCSVMHEAL HNHYTQKSLS LSPGK | 445 |
| Light chain amino acid sequence- | |
| SEQ ID NO: 2 | |
| DIQMTQSPSS VSASVGDRVT ITCKASQDVG TAVAWYQQKP GKAPKLLIYW | 50 |
| ASIRHTGVPS RFSGSGSGTD FTLTISSLQP EDFATYYCHQ FATYTFGGGT | 100 |
| KVEIKRTVAA PSVFIFPPSD EQLKSGTASV VCLLNNFYPR EAKVQWKVDN | 150 |
| ALQSGNSQES VTEQDSKDST YSLSSTLTLS KADYEKHKVY ACEVTHQGLS | 200 |
| SPVTKSFNRG EC | |

In some embodiments, breast cancer involved in the present invention is HER2 expression-positive breast cancer, preferably infiltrating locally advanced or metastatic breast cancer as established by histology and/or cytology and is unresectable. In some embodiments, the patient is a stage IV breast cancer patient. In some embodiments, the patient is less than 65 years old. In some embodiments, the patient is equal to or greater than 65 years old.

In some embodiments, a sample from the breast cancer of the patient is HER2 positive, e.g., HER2 positive based on a fluorescence in situ hybridization (FISH) assay (FISH+) and/or immunohistochemistry (IHC). In some embodiments, a sample from the breast cancer of the patient is IHC2+ or IHC3+. In some embodiments, a sample from the breast cancer of the patient is IHC2+ or FISH+. In some embodiments, a sample from the breast cancer of the patient is IHC3+ or FISH+. In some embodiments, a sample from the breast cancer of the patient is IHC2+ and FISH+. In some embodiments, a sample from the breast cancer of the patient is IHC3+ and FISH+. In some embodiments, a sample from the breast cancer of the patient is IHC3+ and FISH− or not detected.

In some embodiments, a sample from the breast cancer of the patient is estrogen receptor (ER) positive. In some embodiments, a sample from the breast cancer of the patient is estrogen receptor (ER) negative. In some embodiments, a sample from the breast cancer of the patient is progesterone receptor (PR) positive. In some embodiments, a sample from the breast cancer of the patient is progesterone receptor (PR) negative. In some embodiments, a sample from the breast cancer of the patient is ER+ and/or PR+. In some embodiments, a sample from the breast cancer of the patient is ER− and PR−.

In some embodiments, patients for treatment according to the present invention have previously received one or more prior treatments of chemotherapy drugs, targeted therapy, immunotherapy, and endocrine therapy; preferably, they have previously received taxane systemic therapy; or they must have previously received systemic therapy with trastuzumab or a biosimilar thereof at least once.

In some embodiments, the antibody-drug conjugate or medicine of the present invention may be administered intranasally, subcutaneously, intradermally, intramuscularly or intravenously. It is administered at a dose of 2.0 mg/kg every 2 weeks. In some embodiments, the medicine comprises the antibody-drug conjugate and a pharmaceutically acceptable carrier. In some embodiments, administration of the antibody-drug conjugate to the breast cancer patient results in improved progression-free survival (PFS), as compared to administration of capecitabine and lapatinib. In some embodiments, the antibody-drug conjugate is administered as a monotherapy.

Exemplary and non-limiting embodiments of the present disclosure are provided below.

### Embodiment 1

Use of an antibody-drug conjugate (ADC) in the preparation of a medicine for treating a breast cancer patient with liver metastasis, wherein the antibody-drug conjugate has the structure of the general formula Ab-(L-U)ₙ, wherein Ab represents anti-Her2 (Human epidermal growth factor receptor 2) antibody; L represents a linker; U represents a conjugated cytotoxic molecule; and n is an integer from 1 to 8, and represents the number of cytotoxic molecules bound to each antibody, and wherein:
the antibody comprises a heavy chain variable region and a light chain variable region, where the CDR of the heavy chain variable region and/or the CDR of the light chain variable region have the same CDR sequences as Disitamab vedotin;
the linker L comprises Maleimido-Caproyl-Valine-Citrulline-p-Aminobenzyloxy (mc-vc-pAB) and is covalently linked to the antibody by means of sulfhydryl conjugation, and the linking site is the interchain disulfide bond site of the antibody; and
the cytotoxic molecule U comprises MMAE (monomethyl auristatin E).

### Embodiment 2

Use of an antibody-drug conjugate (ADC) in the preparation of a medicine for treating of a breast cancer patient without lung metastasis, wherein the antibody-drug conjugate has the structure of the general formula Ab-(L-U)ₙ, wherein Ab represents anti-Her2 (Human epidermal growth factor receptor 2) antibody; L represents a linker; U represents a conjugated cytotoxic molecule; and n is an integer from 1 to 8, and represents the number of cytotoxic molecules bound to each antibody, and wherein:
the antibody comprises a heavy chain variable region and a light chain variable region, where the CDR of the heavy chain variable region and/or the CDR of the light chain variable region have the same CDR sequences as Disitamab vedotin;
the linker L comprises Maleimido-Caproyl-Valine-Citrulline-p-Aminobenzyloxy (mc-vc-pAB) and is covalently linked to the antibody by means of sulfhydryl conjugation, and the linking site is the interchain disulfide bond site of the antibody; and
the cytotoxic molecule U comprises MMAE (monomethyl auristatin E).

### Embodiment 3

The use according to embodiment 1 or 2, wherein the breast cancer patient is positive for HER2 expression.

### Embodiment 4

The use according to embodiment 3, wherein a sample obtained from the breast cancer of the patient is HER2 positive.

### Embodiment 5

The use according to embodiment 4, wherein the sample obtained from the breast cancer of the patient is HER2 positive based on a fluorescence in situ hybridization (FISH) assay (FISH) and/or immunohistochemistry (IHC) assay.

### Embodiment 6

The use according to embodiment 5, wherein HER2 expression in the sample obtained from the breast cancer of the patient is: IHC3+; IHC2+ or IHC3+; IHC2+ or FISH+; IHC3+ or FISH+; IHC2+ and FISH+; IHC3+ and FISH+; or IHC3+ and FISH− or not detected.

### Embodiment 7

The use according to any one of embodiments 1-6, wherein a sample obtained from the breast cancer of the patient is estrogen receptor (ER) positive and/or progesterone receptor (PR) positive; or wherein a sample obtained from the breast cancer of the patient is ER negative and PR negative.

### Embodiment 8

The use according to any one of embodiments 1-7, wherein the patient has locally advanced or metastatic breast cancer.

### Embodiment 9

The use according to any one of embodiments 1-7, wherein the patient has stage IV breast cancer.

### Embodiment 10

The use according to any one of embodiments 1-9, wherein the patient has unresectable breast cancer.

### Embodiment 11

The use according to embodiment 3, wherein the breast cancer is infiltrating locally advanced or metastatic breast cancer as established by histology and/or cytology and is unresectable.

### Embodiment 12

The use according to any one of embodiments 1-11, wherein the antibody comprises a heavy chain variable (VH) region and a light chain variable (VL) region; wherein the VH region comprises an HCDR1 comprising the amino acid sequence of GYTFTDYY (SEQ ID NO:3), an HCDR2 comprising the amino acid sequence of VNPDHGDS (SEQ ID NO:4), and an HCDR3 comprising the amino acid sequence of ARNYLFDH (SEQ ID NO:5); and wherein the VL region comprises a LCDR1 comprising the amino acid sequence of QDVGTA (SEQ ID NO:6), a LCDR2 comprising the amino acid sequence of WAS (SEQ ID NO:7), and a LCDR3 comprising the amino acid sequence of HQFATYT (SEQ ID NO:8).

### Embodiment 13

The use according to any one of embodiments 1-11, wherein the antibody comprises a heavy chain variable (VH) region and a light chain variable (VL) region; wherein the VH region comprises an HCDR1 comprising the amino acid sequence of DYYIH (SEQ ID NO:11), an HCDR2 comprising the amino acid sequence of RVNPDHGDSYYNQKFKD (SEQ ID NO:12), and an HCDR3 comprising the amino acid sequence of ARNYLFDHW (SEQ ID NO:13); and wherein the VL region comprises a LCDR1 comprising the amino acid sequence of KASQDVGTAVA (SEQ ID NO:14), a LCDR2 comprising the amino acid sequence of WASIRHT (SEQ ID NO:15), and a LCDR3 comprising the amino acid sequence of HQFATYT (SEQ ID NO:8).

### Embodiment 14

The use according to any one of embodiments 1-13, wherein the antibody is a murine, chimeric, or humanized antibody.

### Embodiment 15

The use according to any one of embodiments 1-13, wherein the antibody comprises a heavy chain variable (VH) region and a light chain variable (VL) region; wherein the VH region comprises the amino acid sequence of EVQLVQSGAEVKKPGATVKISCKVSGYTFTDYYIHWVQQAPGKGLEWMGRVNPDH GDSYYNQKFKDKATITADKSTDTAYMELSSLRSEDTAVYFCARNYLFDHWGQGTL VTVSS (SEQ ID NO:9); and wherein the VL region comprises the amino acid sequence of

(SEQ ID NO: 10)
DIQMTQSPSSVSASVGDRVTITCKASQDVGTAVAWYQQKPGKAPKLLIY
WASIRHTGVPSRFSGSGSGTDFTLTISSLQPEDFATYYCHQFATYTFGG
GTKVEIK.

### Embodiment 16

The use according to embodiment 14 or embodiment 15, wherein the antibody is a human IgG antibody.

### Embodiment 17

The use according to embodiment 16, wherein the antibody is a human IgG1, IgG2, or IgG4 antibody.

### Embodiment 18

The use according to any one of embodiments 1-11, wherein the amino acid sequence of the heavy chain of the antibody is shown in SEQ ID NO:1, and the amino acid sequence of the light chain of the antibody is shown in SEQ ID NO:2.

### Embodiment 19

The use according to any one of embodiments 1-11, wherein the antibody-drug conjugate is Disitamab vedotin or a biosimilar thereof.

### Embodiment 20

The use according to any one of embodiments 1-19, wherein the average DAR (i.e., Drug-to-Antibody Ratio) value of the antibody-drug conjugate is any number from 2 to 7.

### Embodiment 21

The use according to embodiment 20, wherein the average DAR value is 4±0.5.

### Embodiment 22

The use according to any one of embodiments 1-21, wherein the patient has previously received one or more prior treatments of chemotherapy drugs, targeted therapy, immunotherapy, and endocrine therapy.

### Embodiment 23

The use according to embodiment 22, wherein the patient has previously received taxane systemic therapy.

### Embodiment 24

The use according to embodiment 22, wherein the patient has previously received systemic therapy with trastuzumab or a biosimilar thereof at least once.

### Embodiment 25

The use according to any one of embodiments 1-24, wherein the medicine is administered intranasally, subcutaneously, intradermally, intramuscularly or intravenously.

### Embodiment 26

The use according to any one of embodiments 1-25, wherein the antibody-drug conjugate is administered at a dose of 2.0 mg/kg every 2 weeks.

### Embodiment 27

The use according to any one of embodiments 1-26, wherein the antibody-drug conjugate is administered as a monotherapy.

### Embodiment 28

The use according to any one of embodiments 1-27, wherein administration of the antibody-drug conjugate to the breast cancer patient results in improved progression-free survival (PFS), as compared to administration of capecitabine and lapatinib.

### Embodiment 29

A method of treating breast cancer, comprising administering to a patient in need thereof a therapeutically effective amount of an antibody-drug conjugate (ADC), wherein the antibody-drug conjugate has the structure of the general formula Ab-(L-U)ₙ, wherein Ab represents an antibody that specifically binds human epidermal growth factor receptor 2 (HER2); L represents a linker; U represents a cytotoxic molecule; and n is an integer from 1 to 8 representing a number of cytotoxic molecule(s) conjugated to each antibody, and wherein:
the antibody comprises a heavy chain variable region and a light chain variable region, where the CDR of the heavy chain variable region and/or the CDR of the light chain variable region have the same CDR sequences as Disitamab vedotin;
the linker L comprises Maleimido-Caproyl-Valine-Citrulline-p-Aminobenzyloxy (mc-vc-pAB) and is covalently linked to the antibody by means of sulfhydryl conjugation, and the linking site is the interchain disulfide bond site of the antibody;
the cytotoxic molecules U comprise MMAE (monomethyl auristatin E); and
the patient has a liver metastasis and/or does not have a lung metastasis.

### Embodiment 30

The method of embodiment 29, wherein the breast cancer patient is positive for HER2 expression.

### Embodiment 31

The method of embodiment 30, wherein a sample obtained from the breast cancer of the patient is HER2 positive.

### Embodiment 32

The method of embodiment 31, wherein the sample obtained from the breast cancer of the patient is HER2 positive based on a fluorescence in situ hybridization (FISH) assay (FISH) and/or immunohistochemistry (IHC) assay.

### Embodiment 33

The method of embodiment 32, wherein HER2 expression in the sample obtained from the breast cancer of the patient is: IHC3+; IHC2+ or IHC3+; IHC2+ or FISH+; IHC3+ or FISH+; IHC2+ and FISH+; IHC3+ and FISH+; or IHC3+ and FISH− or not detected.

### Embodiment 34

The method of any one of embodiments 29-33, wherein a sample obtained from the breast cancer of the patient is estrogen receptor (ER) positive and/or progesterone receptor (PR) positive; or wherein a sample obtained from the breast cancer of the patient is ER negative and PR negative.

### Embodiment 35

The method of any one of embodiments 29-34, wherein the patient has locally advanced or metastatic breast cancer.

### Embodiment 36

The method of any one of embodiments 29-34, wherein the patient has stage IV breast cancer.

### Embodiment 37

The method of any one of embodiments 29-36, wherein the patient has unresectable breast cancer.

### Embodiment 38

The method of any one of embodiments 29-36, wherein the breast cancer is infiltrating locally advanced or metastatic breast cancer as established by histology and/or cytology and is unresectable.

### Embodiment 39

The method of any one of embodiments 29-38, wherein the antibody comprises a heavy chain variable (VH) region and a light chain variable (VL) region; wherein the VH region comprises an HCDR1 comprising the amino acid sequence of GYTFTDYY (SEQ ID NO:3), an HCDR2 comprising the amino acid sequence of VNPDHGDS (SEQ ID NO:4), and an HCDR3 comprising the amino acid sequence of ARNYLFDH (SEQ ID NO:5); and wherein the VL region comprises a LCDR1 comprising the amino acid sequence of QDVGTA (SEQ ID NO:6), a LCDR2 comprising the amino acid sequence of WAS (SEQ ID NO:7), and a LCDR3 comprising the amino acid sequence of HQFATYT (SEQ ID NO:8).

### Embodiment 40

The method of any one of embodiments 29-38, wherein the antibody comprises a heavy chain variable (VH) region and a light chain variable (VL) region; wherein the VH region comprises an HCDR1 comprising the amino acid sequence of DYYIH (SEQ ID NO:11), an HCDR2 comprising the amino acid sequence of RVNPDHGDSYYNQKFKD (SEQ ID NO:12), and an HCDR3 comprising the amino acid sequence of ARNYLFDHW (SEQ ID NO:13); and wherein the VL region comprises a LCDR1 comprising the amino acid sequence of KASQDVGTAVA (SEQ ID NO:14), a LCDR2 comprising the amino acid sequence of WASIRHT (SEQ ID NO:15), and a LCDR3 comprising the amino acid sequence of HQFATYT (SEQ ID NO:8).

### Embodiment 41

The method of any one of embodiments 29-40, wherein the antibody is a murine, chimeric, or humanized antibody.

### Embodiment 42

The method of any one of embodiments 29-40, wherein the antibody comprises a heavy chain variable (VH) region and a light chain variable (VL) region; wherein the VH region comprises the amino acid sequence of EVQLVQSGAEVKKPGATVKISCKVSGYTFTDYYIHWVQQAPGKGLEWMGRVNPDH GDSYYNQKFKDKATITADKSTDTAYMELSSLRSEDTAVYFCARNYLFDHWGQGTL VTVSS (SEQ ID NO:9); and wherein the VL region comprises the amino acid sequence of

(SEQ ID NO: 10)
DIQMTQSPSSVSASVGDRVTITCKASQDVGTAVAWYQQKPGKAPKLLIY
WASIRHTGVPSRFSGSGSGTDFTLTISSLQPEDFATYYCHQFATYTFGG
GTKVEIK.

### Embodiment 43

The method of embodiment 41 or embodiment 42, wherein the antibody is a human IgG antibody.

### Embodiment 44

The method of embodiment 43, wherein the antibody is a human IgG1, IgG2, or IgG4 antibody.

### Embodiment 45

The method of any one of embodiments 29-38, wherein the amino acid sequence of the heavy chain of the antibody is shown in SEQ ID NO:1, and the amino acid sequence of the light chain of the antibody is shown in SEQ ID NO:2.

### Embodiment 46

The method of any one of embodiments 29-38, wherein the antibody-drug conjugate is Disitamab vedotin or a biosimilar thereof.

### Embodiment 47

The method of any one of embodiments 29-46, wherein the average DAR (i.e., Drug-to-Antibody Ratio) value of the antibody-drug conjugate is any number from 2 to 7.

### Embodiment 48

The method of embodiment 47, wherein the average DAR value is 4±0.5.

### Embodiment 49

The method of any one of embodiments 29-48, wherein, prior to administration of the ADC, the patient has previously received one or more prior treatments comprising a chemotherapeutic agent, targeted therapy, immunotherapy, or endocrine therapy.

### Embodiment 50

The method of embodiment 49, wherein, prior to administration of the ADC, the patient has received a taxane systemic therapy.

### Embodiment 51

The method of embodiment 49, wherein, prior to administration of the ADC, the patient has received systemic therapy with trastuzumab or a biosimilar thereof at least once.

### Embodiment 52

The method of any one of embodiments 29-51, wherein the ADC is administered intranasally, subcutaneously, intradermally, intramuscularly, or intravenously.

### Embodiment 53

The method of any one of embodiments 29-52, wherein the ADC is administered at a dose of 2.0 mg/kg every 2 weeks.

### Embodiment 54

The method of any one of embodiments 29-53, wherein the ADC is administered as a monotherapy.

### Embodiment 55

The method of any one of embodiments 29-54, wherein administration of the ADC results in improved progression-free survival (PFS) of the patient, as compared to administration of capecitabine and lapatinib.

### Embodiment 56

The method of any one of embodiments 29-55, wherein the ADC is administered in a pharmaceutical composition comprising the ADC and a pharmaceutically acceptable carrier.

### EXAMPLES

The present invention is further illustrated through the examples below, but it is not intended to limit the scope of the present invention within the scope of the described examples. The experimental methods not specified for the specific conditions in the following Examples are selected according to conventional methods and conditions, or according to the product instructions.

### Example 1

### Overall Data Analysis of the Treatment of Breast Cancer Patients

This study was a parallel-group, randomized, open-label clinical trial to evaluate the efficacy and safety of Disitamab vedotin as compared to treatment with lapatinib and capecitabine in patients with HER2-positive (positive being defined as IHC 3+ or FISH +) locally advanced or metastatic breast cancer. Subjects were randomized 1:1.

The primary endpoint indicator of the study was PFS (progression-free survival), which was evaluated for 6 weeks (±7 days).

The trial has completed the enrollment of 200 patients for treatment.

### Investigational Medical Products

Disitamab vedotin, namely RC48-ADC (average DAR value: 4±0.5), was administered at a dose of 2.0 mg/kg via intravenous drip, once every 2 weeks, 42 days as one treatment cycle.

### Control Treatment Group

Lapatinib was administered at 1250 mg once a day, 21 days as one cycle; and Capecitabine was administered at a total daily dose of 2000 mg/m² for continuous 14 days and rest for 7 days, 21 days as one cycle.

ITT (Intention-To-Treat) analysis of the enrolled 200 patients is shown in Table 1.

TABLE 1

| | Disitamab | Lapatinib + | |
|---|---|---|---|
| | vedotin | Capecitabine | Total |
| | (N = 99) | (N = 101) | (N = 200) |
| Age (years) | | | |
| Cases | 99 | 101 | 200 |
| Mean (standard deviation) | 50.7 (8.84) | 49.5 (10.30) | 50.1 (9.60) |
| Median | 52.0 | 51.0 | 52.0 |
| Minimum, Maximum | 27, 69 | 24, 69 | 24, 69 |
| Age groups, n (%) | | | |
| <65 yrs | 93 (93.9%) | 93 (92.1%) | 186 (93.0%) |
| >=65 yrs | 6 (6.1%) | 8 (7.9%) | 14 (7.0%) |
| ECOG PS, n (%) | | | |
| 0 | 36 (36.4%) | 41 (40.6%) | 77 (38.5%) |
| 1 | 62 (62.6%) | 59 (58.4%) | 121 (60.5%) |
| Absent | 1 (1.0%) | 1 (1.0%) | 2 (1.0%) |
| Course of breast cancer | | | |
| (months) | | | |
| Cases | 99 | 101 | 200 |
| Mean (standard deviation) | 34.443 (33.7590) | 34.214 (30.4258) | 34.327 (32.0382) |
| Median | 22.768 | 24.082 | 23.540 |
| Minimum, Maximum | 2.79, 198.97 | 2.63, 159.24 | 2.63, 198.97 |
| Pathogenic type, n (%) | | | |
| Infiltrating papillary | 0 | 1 (1.0%) | 1 (0.5%) |
| carcinoma | | | |
| Infiltrating duct carcinoma | 72 (72.7%) | 72 (71.3%) | 144 (72.0%) |
| Infiltrating lobular carcinoma | 1 (1.0%) | 1 (1.0%) | 2 (1.0%) |
| Other | 26 (26.3%) | 27 (26.7%) | 53 (26.5%) |
| Current clinical stage, n (%) | | | |
| Stage I | 1 (1.0%) | 0 | 1 (0.5%) |
| Stage IIB | 1 (1.0%) | 1 (1.0%) | 2 (1.0%) |
| Stage III | 1 (1.0%) | 0 | 1 (0.5%) |
| Stage IIIB | 0 | 2 (2.0%) | 2 (1.0%) |
| Stage IIIC | 0 | 5 (5.0%) | 5 (2.5%) |
| Stage IV | 96 (97.0%) | 92 (91.1%) | 188 (94.0%) |
| Absent | 0 | 1 (1.0%) | 1 (0.5%) |
| Metastatic lesions, n (%) | | | |
| None | 1 (1.0%) | 0 | 1 (0.5%) |
| Lymph node | 48 (48.5%) | 52 (51.5%) | 100 (50.0%) |
| Lung | 44 (44.4%) | 52 (51.5%) | 96 (48.0%) |
| Liver | 38 (38.4%) | 43 (42.6%) | 81 (40.5%) |
| Bone | 36 (36.4%) | 33 (32.7%) | 69 (34.5%) |
| Skin | 8 (8.1%) | 5 (5.0%) | 13 (6.5%) |
| Pleura | 7 (7.1%) | 6 (5.9%) | 13 (6.5%) |
| Peritoneum | 2 (2.0%) | 3 (3.0%) | 5 (2.5%) |
| Adrenal gland | 1 (1.0%) | 2 (2.0%) | 3 (1.5%) |
| Heart | 0 | 1 (1.0%) | 1 (0.5%) |
| Other | 30 (30.3%) | 19 (18.8%) | 49 (24.5%) |
| Visceral organ metastasis | 75 (75.8%) | 78 (77.2%) | 153 (76.5%) |
| (IWRS), n (%) | | | |
| Visceral organ metastasis | 77 (77.8%) | 78 (77.2%) | 155 (77.5%) |
| (CRF), n (%) | | | |
| ER/PR, n (%) | | | |
| ER+ or PR+ | 42 (42.4%) | 57 (56.4%) | 99 (49.5%) |
| ER− and PR− | 45 (45.5%) | 40 (39.6%) | 85 (42.5%) |
| ER not detected and PR- | 1 (1.0%) | 0 | 1 (0.5%) |
| Absent | 11 (11.1%) | 4 (4.0%) | 15 (7.5%) |
| IHC/FISH (enrolled), n (%) | | | |
| IHC 2+, FISH+ | 18 (18.2%) | 16 (15.8%) | 34 (17.0%) |
| IHC 3+, FISH− | 0 | 1 (1.0%) | 1 (0.5%) |
| IHC 3+, FISH+ | 14 (14.1%) | 8 (7.9%) | 22 (11.0%) |
| IHC 3+, FISH not detected | 66 (66.7%) | 74 (73.3%) | 140 (70.0%) |
| IHC not detected, FISH+ | 1 (1.0%) | 2 (2.0%) | 3 (1.5%) |
| IHC/FISH (central laboratory), n | | | |
| (%) | | | |
| IHC 1+, FISH not detected | 4 (4.0%) | 1 (1.0%) | 5 (2.5%) |
| IHC 2+, FISH− | 3 (3.0%) | 2 (2.0%) | 5 (2.5%) |
| IHC 2+, FISH+ | 11 (11.1%) | 13 (12.9%) | 24 (12.0%) |
| IHC 2+, not determined | 1 (1.0%) | 1 (1.0%) | 2 (1.0%) |
| IHC 3+, FISH+ | 1 (1.0%) | 1 (1.0%) | 2 (1.0%) |
| IHC 3+, FISH not detected | 79 (79.8%) | 78 (77.2%) | 157 (78.5%) |
| Absent | 0 | 5 (5.0%) | 5 (2.5%) |
| Previous oncology medication | | | |
| treatment, n (%) | | | |
| Chemotherapy | 99 (100%) | 101 (100%) | 200 (100%) |
| Targeted therapy | 97 (98.0%) | 95 (94.1%) | 192 (96.0%) |
| Immunity therapy | 1 (1.0%) | 0 | 1 (0.5%) |
| Endocrine therapy | 42 (42.4%) | 47 (46.5%) | 89 (44.5%) |
| Cell therapy | 0 | 0 | 0 |
| Number of treatment lines | | | |
| received after | | | |
| recurrence/metastasis (IWRS), n | | | |
| (%) | | | |
| <=1 | 85 (85.9%) | 86 (85.1%) | 171 (85.5%) |
| 2 | 14 (14.1%) | 15 (14.9%) | 29 (14.5%) |
| Number of treatment lines | | | |
| received after recurrence/ | | | |
| metastasis (CRF), n (%) | | | |
| <=1 | 83 (83.8%) | 89 (88.1%) | 172 (86.0%) |
| 2 | 16 (16.2%) | 12 (11.9%) | 28 (14.0%) |

Percentage calculations are based on the total number of subjects in each subgroup in the ITT analysis set.
a Survival function estimates, and survival time estimates were obtained using the Kaplan-Meier method, and the error of the survival function was estimated using the Greenwood formula, resulting in 95% CI for different quantile times.
b Considering actual stratification factors: number of previous chemotherapy lines received for advanced disease (≤1 vs. 2), and presence or absence of visceral organ metastasis (yes vs. no).
c The COX proportional hazards model used treatment group and actual stratification factors (number of previous chemotherapy lines received for advanced disease (≤1 vs. 2), and presence or absence of visceral organ metastasis (yes vs. no)) as independent variables to calculate the hazard ratio of RC48-ADC relative to capecitabine+lapatinib, 95% CI, and P value.
d The COX proportional hazards model used treatment group as an independent variable to calculate the hazard ratio, 95% CI, and P value of RC48-ADC relative to capecitabine+lapatinib.
Note: The superscripts a, b, c, d in Tables 2-6 below has the same meanings as defined above.

TABLE 2
Analysis of progression-free survival (PFS).

| | | Capecitabine + |
|---|---|---|
| | Disitamab vedotin | lapatinib |
| | (N = 99) | (N = 101) |
| Disease progression or death, n (%) | 68 (68.7%) | 81 (80.2%) |
| Censoring, n(%) | 31 (31.3%) | 20 (19.8%) |
| Progression-free survival (months)a | | |
| Median (95% CI) | 9.3 (6.9, 12.2) | 7.1 (5.6, 8.3) |
| Stratified Log-Rank test P-value b | 0.1823 | |
| Unstratified Log-Rank test P value | 0.2378 | |
| COX proportional hazards model | | |
| (considering stratification factors) c | | |
| Hazard ratio (95% CI) | 0.82 (0.59, 1.13) | |
| P value | 0.2314 | |
| COX proportional hazards model | | |
| (without considering stratification | | |
| factors)d | | |
| Hazard ratio (95% CI) | 0.82 (0.60, 1.14) | |
| P value | 0.2422 | |

Percentage calculations are based on the total number of subjects in each subgroup in the ITT analysis set.

The overall data analysis showed that when comparing the Disitamab vedotin experimental group and the control group (lapatinib+capecitabine), the progression-free survival data were 9.3 months and 7.1 months, respectively. Compared with the control group, the experimental group did not show a statistically significant advantage in the overall sample of breast cancer patients.

### Example 2

### Analysis of Progression-Free Survival (PFS) in a Liver Metastasis Subgroup

This example is intended to compare the effect of the Disitamab vedotin treatment group and the control treatment group (lapatinib+capecitabine) on the progression-free survival (PFS) in the liver metastasis subgroup. There were 99 subjects in the Disitamab vedotin treatment group, including 38 cases with liver metastasis occurred and 61 cases without liver metastasis occurred; and there were 101 research subjects in the control treatment group, including 43 cases with liver metastasis occurred and 58 cases without liver metastasis occurred. The data analysis of treatment outcomes showed (see Table 3) that the progression-free survival time (median) of the patients without liver metastasis in the Disitamab vedotin treatment group was 7.0 months, and the progression-free survival time (median) of the patients without liver metastasis in the control treatment group was 9.0 months. The progression-free survival time (median) of the patients without liver metastasis in the Disitamab vedotin treatment group was shorter than that in the control treatment group by 2.0 months. However, we surprisingly found that the progression-free survival time (median) of the patients with liver metastasis in the Disitamab vedotin treatment group was 12.5 months, and the progression-free survival time (median) of the patients with liver metastasis in the control treatment group was only 5.6 months. The progression-free survival time (median) of the patients with liver metastasis in the Disitamab vedotin treatment group was significantly longer than the progression-free survival time (median) of the patients with liver metastasis in the control treatment group by 6.9 months. Compared with the control treatment group, the Disitamab vedotin treatment group can significantly improve the disease progression-free time and survival time of the patients with liver metastasis, which has extremely high clinical value.

TABLE 3
Evaluation of progression-free survival (PFS) in
a liver metastasis subgroup (ITT analysis set).
Liver metastasis: Yes

| | Disitamab | Capecitabine + |
|---|---|---|
| | vedotin | lapatinib |
| | (N = 38) | (N = 43) |
| Disease progression or death, | 22 (57.9%) | 36 (83.7%) |
| n (%) | | |
| Censoring, n(%) | 16 (42.1%) | 7 (16.3%) |
| Progression-free survival time | | |
| (months)^{a} | | |
| Median (95% CI) | 12.5 (7.1, 14.1) | 5.6 (4.1, 6.9) |
| Stratified Log-Rank test P-value ^{b} | 0.0011 | |
| Unstratified Log-Rank test P value | 0.0009 | |
| COX proportional hazards model | | |
| (considering stratification | | |
| factors)^{c} | | |
| Hazard ratio (95% CI) | 0.42 (0.24, 0.72) | |
| P value | 0.0017 | |
| COX proportional hazards model | | |
| (without considering | | |
| stratification factors)^{d} | | |
| Hazard ratio (95% CI) | 0.41 (0.24, 0.71) | |
| P value | 0.0014 | |

Liver metastasis: No

| | Disitamab | Capecitabine + |
|---|---|---|
| | vedotin | lapatinib |
| | (N = 61) | (N = 58) |
| Disease progression or death, | 46 (75.4%) | 45 (77.6%) |
| n (%) | | |
| Censoring, n(%) | 15 (24.6%) | 13 (22.4%) |
| Progression-free survival time | | |
| (months)^{a} | | |
| Median (95% CI) | 7.0 (5.5, 9.9) | 9.0 (6.9, 12.2) |
| Stratified Log-Rank test P-value ^{b} | 0.2356 | |
| Unstratified Log-Rank test P value | 0.2723 | |
| COX proportional hazards model | | |
| (considering stratification | | |
| factors)^{c} | | |
| Hazard ratio (95% CI) | 1.27 (0.83, 1.92) | |
| P value | 0.2688 | |
| COX proportional hazards model | | |
| (without considering | | |
| stratification factors)^{d} | | |
| Hazard ratio (95% CI) | 1.26 (0.83, 1.91) | |
| P value | 0.2757 | |

Percentage calculations are based on the total number of subjects in each subgroup in the ITT analysis set.

### Example 3

### Analysis of Progression-Free Survival (PFS) in a Lung Metastasis Subgroup

This example is intended to compare the effect of the Disitamab vedotin treatment group and the control treatment group (lapatinib+capecitabine) on the progression-free survival (PFS) in the lung metastasis subgroup. There were 99 research subjects in the Disitamab vedotin treatment group, including 44 cases with lung metastasis and 55 cases without lung metastasis; and there were 101 research subjects in the control treatment group, including 52 cases with lung metastasis and 49 cases without lung metastasis. The data analysis of treatment outcomes showed (see Table 4) that the median progression-free survival time of the patients with lung metastasis in the Disitamab vedotin treatment group was 8.2 months, and the progression-free survival time (median) of the patients with lung metastasis in the control treatment group was 8.3 months, there being no significant difference between the two. However, we also surprisingly found that the progression-free survival time (median) of the patients without lung metastasis in the Disitamab vedotin treatment group was 10.9 months, and the progression-free survival time (median) of the patients without lung metastasis in the control treatment group was only 5.6 months. The progression-free survival time (median) of the patients without lung metastasis in the Disitamab vedotin treatment group was significantly longer than the progression-free survival time (median) of the patients without lung metastasis in the control treatment group by 5.3 months. Compared with the control treatment group, the Disitamab vedotin treatment group can significantly improve the disease progression-free time and survival time of the patients, which has extremely high clinical value.

TABLE 4
Evaluation of progression-free survival (PFS) in
a lung metastasis subgroup (ITT analysis set)
Lung metastasis: Yes

| | Disitamab | Capecitabine + |
|---|---|---|
| | vedotin | lapatinib |
| | (N = 44) | (N = 52) |
| Disease progression or death, n (%) | 34 (77.3%) | 43 (82.7%) |
| Censoring, n(%) | 10 (22.7%) | 9 (17.3%) |
| Progression-free survival time | | |
| (months)^{a} | | |
| Median (95% CI) | 8.2 (5.6, 12.2) | 8.3 (6.9, 11.0) |
| Stratified Log-Rank test P-value ^{b} | 0.8000 | |
| Unstratified Log-Rank test P value | 0.8388 | |
| COX proportional hazards model | | |
| (considering stratification factors)^{c} | | |
| Hazard ratio (95% CI) | 1.04 (0.66, 1.65) | |
| P value | 0.8523 | |
| COX proportional hazards model | | |
| (without considering | | |
| stratification factors)^{d} | | |
| Hazard ratio (95% CI) | 1.05 (0.67, 1.65) | |
| P value | 0.8395 | |

Lung metastasis: No

| | Disitamab | Capecitabine + |
|---|---|---|
| | vedotin | lapatinib |
| | (N = 55) | (N = 49) |
| Disease progression or death, n (%) | 34 (61.8%) | 38 (77.6%) |
| Censoring, n(%) | 21 (38.2%) | 11 (22.4%) |
| Progression-free survival time | | |
| (months)^{a} | | |
| Median (95% CI) | 10.9 (5.7, 13.7) | 5.6 (4.2, 7.6) |
| Stratified Log-Rank test P-value ^{b} | 0.0581 | |
| Unstratified Log-Rank test P value | 0.0940 | |
| COX proportional hazards model | | |
| (considering stratification factors)^{c} | | |
| Hazard ratio (95% CI) | 0.61 (0.37, 0.99) | |
| P value | 0.0439 | |
| COX proportional hazards model | | |
| (without considering | | |
| stratification factors)^{d} | | |
| Hazard ratio (95% CI) | 0.68 (0.42, 1.07) | |
| P value | 0.0976 | |

Percentage calculations are based on the total number of subjects in each subgroup in the ITT analysis set.

### Example 4

### Analysis of Progression-Free Survival (PFS) in a Bone Metastasis Subgroup

This example is intended to compare the effect of the Disitamab vedotin treatment group and the control treatment group (lapatinib+capecitabine) on the progression-free survival (PFS) in the bone metastasis subgroup. There were 99 research subjects in the Disitamab vedotin treatment group, including 36 cases with bone metastasis and 63 cases without bone metastasis; and there were 101 research subjects in the control treatment group, including 33 cases with bone metastasis and 68 cases without bone metastasis. The data analysis of treatment outcomes showed (see Table 5) that the progression-free survival time (median) of the patients with bone metastasis in the Disitamab vedotin treatment group was 7.8 months, and the progression-free survival time (median) of patients with bone metastasis in the control treatment group was 6.0 months. The progression-free survival time (median) of the patients with bone metastasis in the Disitamab vedotin treatment group was slightly longer than in the control treatment group, but there was no very significant difference between the two. The progression-free survival time (median) of the patients without bone metastasis in the Disitamab vedotin treatment group was 9.6 months, and the progression-free survival time (median) of the patients without bone metastasis in the control treatment group was 8.0 months. The progression-free survival time (median) of the patients without bone metastasis in the Disitamab vedotin treatment group was slightly longer than that in the control treatment group (by 1.6 months), but there was no very significant difference between the two, and patient benefits were limited.

TABLE 5
Evaluation of progression-free survival (PFS) in
a bone metastasis subgroup (ITT analysis set)
Bone metastasis: Yes

| | Disitamab | Capecitabine + |
|---|---|---|
| | vedotin | lapatinib |
| | (N = 36) | (N = 33) |
| Disease progression or death, n (%) | 28 (77.8%) | 28 (84.8%) |
| Censoring, n(%) | 8 (22.2%) | 5 (15.2%) |
| Progression-free survival time | | |
| (months)^{a} | | |
| Median (95% CI) | 7.8 (2.8, 12.4) | 6.0 (4.2, 11.0) |
| Stratified Log-Rank test P-value ^{b} | 0.9291 | |
| Unstratified Log-Rank test P value | 0.8550 | |
| COX proportional hazards model | | |
| (considering stratification factors)^{c} | | |
| Hazard ratio (95% CI) | 1.02 (0.60, 1.74) | |
| P value | 0.9413 | |
| COX proportional hazards model | | |
| (without considering stratification | | |
| factors)^{d} | | |
| Hazard ratio (95% CI) | 0.95 (0.56, 1.61) | |
| P value | 0.8558 | |

Bone metastasis: No

| | Disitamab | Capecitabine + |
|---|---|---|
| | vedotin | lapatinib |
| | (N = 63) | (N = 68) |
| Disease progression or death, n (%) | 40 (63.5%) | 53 (77.9%) |
| Censoring, n(%) | 23 (36.5%) | 15 (22.1%) |
| Progression-free survival time | | |
| (months)^{a} | | |
| Median (95% CI) | 9.6 (6.9, 13.1) | 8.0 (5.7, 9.0) |
| Stratified Log-Rank test P-value ^{b} | 0.1191 | |
| Unstratified Log-Rank test P value | 0.1926 | |
| COX proportional hazards model | | |
| (considering stratification factors)^{c} | | |
| Hazard ratio (95% CI) | 0.76 (0.50, 1.15) | |
| P value | 0.1919 | |
| COX proportional hazards model | | |
| (without considering | | |
| stratification factors)^{d} | | |
| Hazard ratio (95% CI) | 0.76 (0.50, 1.15) | |
| P value | 0.1966 | |

Percentage calculations are based on the total number of subjects in each subgroup in the ITT analysis set.

### Example 5

### Analysis of Progression-Free Survival (PFS) in a Viscera Metastasis Subgroup

This example is intended to compare the effect of the Disitamab vedotin treatment group and the control treatment group (lapatinib+capecitabine) on the progression-free survival (PFS) in the viscera metastasis subgroup (similarly hereinafter). There were 99 subjects in the Disitamab vedotin treatment group, including 77 cases with viscera metastasis and 22 cases without viscera metastasis; and there were 101 research subjects in the control treatment group, including 78 cases with viscera metastasis and 23 cases without viscera metastasis. The data analysis of treatment outcomes showed (see Table 6) that the progression-free survival time (median) of the patients with viscera metastasis in the Disitamab vedotin treatment group was 9.3 months, and the progression-free survival time (median) of the patients with viscera metastasis in the control treatment group was 6.9 months. The progression-free survival time (median) in the patients with viscera metastasis in the Disitamab vedotin treatment group was longer than that in the control treatment group by 2.4 months, but the hazard ratio test did not show statistical differences between the two. The progression-free survival time (median) of the patients without viscera metastasis in the Disitamab vedotin treatment group was 9.6 months and the progression-free survival time (median) of the patients without viscera metastasis in the control treatment group was 8.1 months. The progression-free survival time (median) of the patients without viscera metastasis in the Disitamab vedotin treatment group was slightly longer than that in the control treatment group (by 1.5 months), but there was also no statistical difference shown between the two.

TABLE 6
Evaluation of progression-free survival (PFS) in
a viscera metastasis subgroup (ITT analysis set)
Viscera metastasis (CRF): Yes

| | Disitamab | Capecitabine + |
|---|---|---|
| | vedotin | lapatinib |
| | (N = 77) | (N = 78) |
| Disease progression or death, n (%) | 53 (68.8%) | 64 (82.1%) |
| Censoring, n(%) | 24 (31.2%) | 14 (17.9%) |
| Progression-free survival time | | |
| (months)a | | |
| Median (95% CI) | 9.3 (6.9, 12.2) | 6.9 (5.6, 8.3) |
| Stratified Log-Rank test P-value b | 0.1319 | |
| Unstratified Log-Rank test P value | 0.1439 | |
| COX proportional hazards | | |
| model (considering | | |
| stratification factors)c | | |
| Hazard ratio (95% CI) | 0.76 (0.53, 1.10) | |
| P value | 0.1485 | |
| COX proportional hazards model | | |
| (without considering stratification | | |
| factors)d | | |
| Hazard ratio (95% CI) | 0.76 (0.53, 1.10) | |
| P value | 0.1481 | |

Viscera metastasis (CRF): No

| | Disitamab | Capecitabine + |
|---|---|---|
| | vedotin | lapatinib |
| | (N = 22) | (N = 23) |
| Disease progression or death, n (%) | 15 (68.2%) | 17 (73.9%) |
| Censoring, n(%) | 7 (31.8%) | 6 (26.1%) |
| Progression-free survival time | | |
| (months)a | | |
| Median (95% CI) | 9.6 (4.3, 14.2) | 8.1 (5.4, 13.8) |
| Stratified Log-Rank test P-value b | 0.9879 | |
| Unstratified Log-Rank test P value | 0.9533 | |
| COX proportional hazards | | |
| model (considering | | |
| stratification factors)c | | |
| Hazard ratio (95% CI) | 0.99 (0.49, 2.00) | |
| P value | 0.9680 | |
| COX proportional hazards model | | |
| (without considering stratification | | |
| factors)d | | |
| Hazard ratio (95% CI) | 1.02 (0.51, 2.05) | |
| P value | 0.9535 | |

Percentage calculations are based on the total number of subjects in each subgroup in the ITT analysis set.

Table 7 presents a summary of the progression-free survival times in different metastatic site subgroups for the Disitamab vedotin treatment group and the control treatment group (lapatinib+capecitabine). From the table, it was observed that:

In breast cancer patients without liver metastasis, the progression-free survival time in the Disitamab vedotin treatment group was 7.0 months, and the progression-free survival time in the capecitabine+lapatinib group was 9.0 months. Compared with capecitabine+lapatinib, the progression-free survival time of the breast cancer patients without liver metastasis in the Disitamab vedotin treatment group was shorter by 2.0 months. In breast cancer patients with liver metastasis, the progression-free survival time in the Disitamab vedotin treatment group was 12.5 months, and the progression-free survival time in the capecitabine+lapatinib group was 5.6 months. Compared with capecitabine+lapatinib, the Disitamab vedotin treatment group significantly prolonged the progression-free survival time by 6.9 months, i.e., compared with capecitabine+lapatinib, the Disitamab vedotin treatment group had a 58% chance to reduce the relative risk of disease progression or death in the breast cancer patients with liver metastasis (hazard ratio HR=0.42). This had great significance in clinical application, and greatly prolonged the disease progression-free time and survival time of the patients.

In breast cancer patients with lung metastasis, the progression-free survival time in the Disitamab vedotin treatment group was 8.2 months, and the progression-free survival time in the capecitabine+lapatinib group was 8.3 months. Compared with capecitabine+lapatinib, the progression-free survival time of the breast cancer patients with lung metastasis occurred in the Disitamab vedotin treatment group was shorter by 0.1 months. In breast cancer patients without lung metastasis, the progression-free survival time in the Disitamab vedotin treatment group was 10.9 months and the progression-free survival time in the capecitabine+lapatinib group was 5.6 months. Compared with capecitabine+lapatinib, the Disitamab vedotin treatment group significantly prolonged the progression-free survival time by 5.3 months, i.e., compared with capecitabine+lapatinib, the Disitamab vedotin treatment group had a 39% chance to reduce the relative risk of disease progression or death in the breast cancer patients without lung metastasis (hazard ratio HR=0.61). This had great significance in clinical application, and greatly prolonged the disease progression-free time and survival time of the patients.

In breast cancer patients without bone metastasis, the progression-free survival time in the Disitamab vedotin treatment group was 9.6 months, and the progression-free survival time in the capecitabine+lapatinib group was 8.0 months; and in breast cancer patients with bone metastasis, the progression-free survival time in the Disitamab vedotin treatment group was 7.8 months, and the progression-free survival time in the capecitabine+lapatinib group was 6.0 months. Although in the two groups, the progression-free survival times in the Disitamab vedotin treatment groups were longer than that in the capecitabine+lapatinib groups, but the prolonged times had no statistically significant difference.

Similarly, in the viscera group, the progression-free survival time of the breast cancer patients with viscera metastasis in the Disitamab vedotin treatment group was 9.3 months, and the progression-free survival time in the capecitabine+lapatinib group was 6.9 months; and in breast cancer patients without viscera metastasis, the progression-free survival time in the Disitamab vedotin treatment group was 9.6 months, and the progression-free survival time in the capecitabine+lapatinib group was 8.1 months. Although in the two groups, the progression-free survival times in the Disitamab vedotin treatment groups were longer than that in the capecitabine+lapatinib groups, but the prolonged times had no statistically significant difference.

That is to say, Disitamab vedotin can effectively prolong the disease progression-free time and survival time of breast cancer patients with liver metastasis or breast cancer patients without lung metastasis, and the effect is non-obvious; i.e., the application of Disitamab vedotin in the subgroup of breast cancer patients with liver metastasis and breast cancer patients without lung metastasis has outstanding substantive features and significant progress, and can clinically produce huge social benefits and provide patients with more precise treatment options, thereby effectively reducing the overall medical burden of the society.

TABLE 7
Summary of progression-free survival in subgroups

| | | Progression-free survival | | | |
|---|---|---|---|---|---|
| | | time (months) | | | |
| | | | Non- | | |
| | | Investigational | Investigative | | |
| | | Medical | Medical | | |
| | | Products | Products | | |
| Metastasis | Metastasis | Disitamab | Capecitabine + | Hazard | |
| location | situation | vedotin | Lapatinib | ratio | Remark |
| Lung | Yes | 8.2 | 8.3 | 1.04 | — |
| | No | 10.9 | 5.6 | 0.61 | Disitamab |
| | | | | | vedotin had a |
| | | | | | 39% chance of |
| | | | | | reducing the |
| | | | | | relative risk of |
| | | | | | disease |
| | | | | | progression or |
| | | | | | death in |
| | | | | | patients. |
| Liver | Yes | 12.5 | 5.6 | 0.42 | Disitamab |
| | | | | | vedotin had a |
| | | | | | 58% chance of |
| | | | | | reducing the |
| | | | | | relative risk of |
| | | | | | disease |
| | | | | | progression or |
| | | | | | death in |
| | | | | | patients. |
| | No | 7.0 | 9.0 | 1.27 | — |
| Bone | Yes | 7.8 | 6.0 | 1.02 | — |
| | No | 9.6 | 8.0 | 0.76 | Disitamab |
| | | | | | vedotin had a |
| | | | | | 24% chance of |
| | | | | | reducing the |
| | | | | | relative risk of |
| | | | | | disease |
| | | | | | progression or |
| | | | | | death in |
| | | | | | patients. |
| Viscera | Yes | 9.3 | 6.9 | 0.99 | — |
| | No | 9.6 | 8.1 | 0.76 | Disitamab |
| | | | | | vedotin had a |
| | | | | | 24% chance of |
| | | | | | reducing the |
| | | | | | relative risk of |
| | | | | | disease |
| | | | | | progression or |
| | | | | | death in |
| | | | | | patients. |

The invention has been exemplified by specific Examples. However, those skilled in the art will appreciate that the present invention is not limited to the specific embodiments. Various modifications or variations can be made within the scope of the present invention, and various technical features mentioned throughout the present specification can be combined with each other without deviating from the spirit and scope of the present invention. Such modifications and variations are all within the scope of the present invention.

1. A method for treating a breast cancer patient with liver metastasis, comprising administering to the patient an effective amount of an antibody-drug conjugate (ADC), wherein the antibody-drug conjugate has the structure of the general formula Ab-(L-U)ₙ, wherein Ab represents an anti-Her2 (Human epidermal growth factor receptor 2) antibody; L represents a linker; U represents a conjugated cytotoxic molecule; and n is an integer from I to 8, and represents the number of cytotoxic molecules bound to each antibody, and wherein:
the linker L comprises Maleimido-Caproyl-Valine-Citrulline-p-Aminobenzyloxy (mcvc-pAB) and is covalently linked to the antibody by means of sulfhydryl conjugation, and the linking site is the interchain disulfide bond site of the antibody; and
the cytotoxic molecule U comprises MMAE (monomethyl auristatin E);
wherein the antibody comprises a heavy chain variable (VH) region and a light chain variable (VL) region;
(a) wherein the VH region comprises an HCDR1 comprising the amino acid sequence of DYYIH (SEQ ID NO:11), an HCDR2 comprising the amino acid sequence of RVNPDHGDSYYNQKFKD (SEQ ID NO:12), and an HCDR3 comprising the amino acid sequence of ARNYLFDHW (SEQ ID NO:13); and wherein the VL region comprises a LCDR1 comprising the amino acid sequence of KASQDVGTAVA (SEQ ID NO:14), a LCDR2 comprising the amino acid sequence of WASIRHT (SEQ ID NO:15), and a LCDR3 comprising the amino acid sequence of HQFATYT (SEQ ID NO:8), or
(b) wherein the VH region comprises an HCDR1 comprising the amino acid sequence of GYTFTDYY (SEQ ID NO:3), an HCDR2 comprising the amino acid sequence of VNPDHGDS (SEQ ID NO:4), and an HCDR3 comprising the amino acid sequence of ARNYLFDH (SEQ ID NO:5); and wherein the VL region comprises a LCDR1 comprising the amino acid sequence of QDVGTA (SEQ ID NO:6), a LCDR2 comprising the amino acid sequence of WAS (SEQ ID NO:7), and a LCDR3 comprising the amino acid sequence of HQFATYT (SEQ ID NO:8).

2. A method for treating a breast cancer patient without lung metastasis, comprising administering to the patient an effective amount of an antibody-drug conjugate (ADC), wherein the antibody-drug conjugate has the structure of the general formula Ab-(L-U)ₙ, wherein Ab represents an anti-Her2 (Human epidermal growth factor receptor 2) antibody; L represents a linker; U represents a conjugated cytotoxic molecule; and n is an integer from 1 to 8, and represents the number of cytotoxic molecules bound to each antibody, and wherein:
the linker L comprises Maleimido-Caproyl-Valine-Citrulline-p-Aminobenzyloxy (mcvc-pAB) and is covalently linked to the antibody by means of sulfhydryl conjugation, and the linking site is the interchain disulfide bond site of the antibody; and
the cytotoxic molecule U comprises MMAE (monomethyl auristatin E);
wherein the antibody comprises a heavy chain variable (VH) region and a light chain variable (VL) region;
(a) wherein the VH region comprises an HCDR1 comprising the amino acid sequence of DYYIH (SEQ ID NO:11), an HCDR2 comprising the amino acid sequence of RVNPDHGDSYYNQKFKD (SEQ ID NO:12), and an HCDR3 comprising the amino acid sequence of ARNYLFDHW (SEQ ID NO:13); and wherein the VL region comprises a LCDR1 comprising the amino acid sequence of KASQDVGTAVA (SEQ ID NO:14), a LCDR2 comprising the amino acid sequence of WASIRHT (SEQ ID NO:15), and a LCDR3 comprising the amino acid sequence of HQFATYT (SEQ ID NO:8), or
(b) wherein the VH region comprises an HCDR1 comprising the amino acid sequence of GYTFTDYY (SEQ ID NO:3), an HCDR2 comprising the amino acid sequence of VNPDHGDS (SEQ ID NO:4), and an HCDR3 comprising the amino acid sequence of ARNYLFDH (SEQ ID NO:5); and wherein the VL region comprises a LCDR1 comprising the amino acid sequence of QDVGTA (SEQ ID NO:6), a LCDR2 comprising the amino acid sequence of WAS (SEQ ID NO:7), and a LCDR3 comprising the amino acid sequence of HQFATYT (SEQ ID NO:8).

3. The method according to claim 1, wherein the breast cancer patient is positive for HER2 expression.

4. The method according to claim 3, wherein a sample obtained from the breast cancer of the patient is HER2 positive.

5. The method according to claim 4, wherein the sample obtained from the breast cancer of the patient is HER2 positive based on a fluorescence in situ hybridization (FISH) assay (FISH) and/or immunohistochemistry (IHC) assay.

6. The method according to claim 5, wherein HER2 expression in the sample obtained from the breast cancer of the patient is: IHC3+; IHC2+ or IHC3+; IHC2+ or FISH+; IHC3+ or FISH+; IHC2+ and FISH+; IHC3+ and FISH+; or IHC3+ and FISH− or not detected.

7. The method according to claim 1, wherein a sample obtained from the breast cancer of the patient is estrogen receptor (ER) positive and/or progesterone receptor (PR) positive; or wherein a sample obtained from the breast cancer of the patient is ER negative and PR negative.

8. The method according to claim 1, wherein the patient has locally advanced or metastatic breast cancer.

9. The method according to claim 1, wherein the patient has stage IV breast cancer.

10. The method according to claim 1, wherein the patient has unresectable breast cancer.

11. The method according to claim 3, wherein the breast cancer is infiltrating locally advanced or metastatic breast cancer as established by histology and/or cytology and is unresectable.

12-13. (canceled)

14. The method according to claim 1, wherein the antibody is a murine, chimeric, or humanized antibody.

15. The method according to claim 1, wherein the antibody comprises a heavy chain variable (VH) region and a light chain variable (VL) region; wherein the VH region comprises the amino acid sequence of EVQLVQSGAEVKKPGATVKISCKVSGYTFTDYYIHWVQQAPGKGLEWMGRVNPDH GDSYYNQKFKDKATITADKSTDTAYMELSSLRSEDTAVYFCARNYLFDHWGQGTL VTVSS (SEQ NO:9); and wherein the VL region comprises the amino acid sequence of
(SEQ ID NO: 10)
DIQMTQSPSSVSASVGDRVTITCKASQDVGTAVAWYQQKPGKAPKLLIY
WASIRHTGVPSRFSGSGSGTDFTLTISSLQPEDFATYYCHQFATYTFGG
GTKVEIK.

16. The method according to claim 14, wherein the antibody is a human IgG antibody.

17. The method according to claim 16, wherein the antibody is a human IgG1, IgG2, or IgG4 antibody.

18. The method according to claim 1, wherein the amino acid sequence of the heavy chain of the antibody is shown in SEQ ID NO:1, and the amino acid sequence of the light chain of the antibody is shown in SEQ ID NO:2.

19. The method according to claim 1, wherein the antibody-drug conjugate is Disitamab vedotin or a biosimilar thereof.

20. The method according to claim 1, wherein the average DAR (i.e., Drug-to-Antibody Ratio) value of the antibody-drug conjugate is any number from 2 to 7.

21. The method according to claim 20, wherein the average DAR value is 4±0.5.

22. The method according to claim 1, wherein the patient has previously received one or more prior treatments of chemotherapy drugs, targeted therapy, immunotherapy, and/or endocrine therapy.

23. The method according to claim 22, wherein the patient has previously received taxane systemic therapy.

24. The method according to claim 22, wherein the patient has previously received systemic therapy with trastuzumab or a biosimilar thereof at least once.

25. The method according to claim 1, wherein the medicine is administered intranasally, subcutaneously, intradermally, intramuscularly or intravenously.

26. The method according to claim 25, wherein the antibody-drug conjugate is administered at a dose of 2.0 mg/kg every 2 weeks.

27. The method according to claim 25, wherein the antibody-drug conjugate is administered as a monotherapy.

28. The method according to claim 25, wherein administration of the antibody-drug conjugate to the breast cancer patient results in improved progression-free survival (PFS), as compared to administration of capecitabine and lapatinib.


SEQUENCE LISTING (15 sequences)

SEQ ID NO: 1 - protein, 445 aa, synthetic construct
  REGION 1..445: note = Synthetic Construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 212 aa, synthetic construct
  REGION 1..212: note = Synthetic Construct
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 8 aa, synthetic construct
  REGION 1..8: note = Synthetic Construct
GYTFTDYY

SEQ ID NO: 4 - protein, 8 aa, synthetic construct
  REGION 1..8: note = Synthetic Construct
VNPDHGDS

SEQ ID NO: 5 - protein, 8 aa, synthetic construct
  REGION 1..8: note = Synthetic Construct
ARNYLFDH

SEQ ID NO: 6 - protein, 6 aa, synthetic construct
  REGION 1..6: note = Synthetic Construct
QDVGTA

SEQ ID NO: 7 - skipped

SEQ ID NO: 8 - protein, 7 aa, synthetic construct
  REGION 1..7: note = Synthetic Construct
HQFATYT

SEQ ID NO: 9 - protein, 115 aa, synthetic construct
  REGION 1..115: note = Synthetic Construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSS

SEQ ID NO: 10 - protein, 105 aa, synthetic construct
  REGION 1..105: note = Synthetic Construct
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIK

SEQ ID NO: 11 - protein, 5 aa, synthetic construct
  REGION 1..5: note = Synthetic Construct
DYYIH

SEQ ID NO: 12 - protein, 17 aa, synthetic construct
  REGION 1..17: note = Synthetic Construct
RVNPDHGDSYYNQKFKD

SEQ ID NO: 13 - protein, 9 aa, synthetic construct
  REGION 1..9: note = Synthetic Construct
ARNYLFDHW

SEQ ID NO: 14 - protein, 11 aa, synthetic construct
  REGION 1..11: note = Synthetic Construct
KASQDVGTAVA

SEQ ID NO: 15 - protein, 7 aa, synthetic construct
  REGION 1..7: note = Synthetic Construct
WASIRHT